(19) United States
(12) Patent Application Publication
(10) Pub. No.: US 2008/0014645 A1
(43) Pub. Date: Jan. 17, 2008

(54) METHOD FOR REDUCING PSA LEVELS

(76) Inventor: HARRY FISCH, SCARSDALE, NY (US)

Correspondence Address: NORRIS, MCLAUGHLIN & MARCUS, PA, 875 THIRD AVENUE, 18TH FLOOR, NEW YORK, NY, 10022, US

(21) Appl. No.: 11/859,209

(22) Filed: Sep. 21, 2007

Related U.S. Application Data
(62) Division of application No. 10/381,225, filed on Mar. 21, 2003.
(60) Provisional application No. 60/340,909, filed on Oct. 29, 2001.
(60) Provisional application No. 60/351,157, filed on Jan. 23, 2002.

(51) Int. Cl. G01N 33/48

(52) U.S. Cl. 436/63

(57) ABSTRACT

A method for reducing an antigen indicator of prostate cancer and for reducing the need for biopsies in men suspected of having prostate cancer and a method for treating patients with elevated PSA levels. In one method, the level of an antigen indicator of prostate cancer is measured and for an above normal level of the antigen indicator, an effective amount of an anti-inflammatory, or a combination of the anti-inflammatory and an antibiotic, is administered and the level of the antigen indicator is remeasured to determine if the level is normal or reduced, whereby a biopsy may not be indicated.

### CROSS REFERENCE TO RELATED APPLICATIONS

This application claims priority to PCT/US02/29713 filed Sep. 19, 2002 and now pending which claims priority to U.S. Provisional Patent Application No. 60/340,909 filed Oct. 29, 2001 and now expired and U.S. Provisional Patent Application No. 60/351,157 filed Jan. 23, 2002 and now expired.

### BACKGROUND OF THE INVENTION

The present invention relates to methods for reducing PSA levels and the need for a biopsy in men suspected of having prostate cancer and for treating patients with elevated PSA levels.

Prostate specific antigen (PSA) is a low molecular weight glycoprotein of approximately 30 kilodaltons (kD) produced mainly by prostatic glandular epithelial cells. PSA is a member of the human tissue kallikrein gene family, which consists of three genes, hKLK1, hKLK2, and hKLK3. The protein product of these genes are pancreatic-kidney kallikrein (hK1), human glandular kallikrein (hK2) (previously known as hGK-1), and PSA (hK3). PSA is a serine protease and exhibits a chymotrypsin-like specificity cleaving at a hydrophobic residue (see Chu et al., J. Urol. 141:152-56 (1989)). The other two kallikreins have trypsin-like specificity. These three genes are located on chromosome 19. The expression of both PSA and hK2 are under androgen control. The protein for hK2 has not been isolated, and its potential clinical utility has not been determined (McCormack et al., Urology 45:729-44 (1995).

PSA is produced in the prostate and occurs in high concentrations in prostate and seminal fluid. It also occurs in urine and in serum. The concentration of serum PSA in the normal male increases with age. Increased levels of serum PSA are found in benign prostatic hyperplasia (BPH), prostate inflammation and prostate cancer. The expression of PSA in most females is negative or very low. While PSA isolated from seminal fluid or prostatic tissue occurs predominately as the 30 kD form of PSA, PSA in the serum occurs in various forms. A small portion of serum PSA occurs as low molecular weight or free PSA. The majority of immunodetectable PSA occurs as a 100 kD complex of PSA and alpha-1-antichymotrypsin (ACT) otherwise known as complexed PSA (PSA-ACT). Another PSA complex formed with alpha-2-macrogobulin is not detectable by current immunoassays. PSA complexes with these protease inhibitors through its serine protease active site. Presumably, the free PSA in serum is inactive PSA, since serum contains an excess of protease inhibitors (McCormack et al., Urology 45:729-44 (1995), Partin et al., J. Urol. 152:1358-68(1994), Lilja et al., Clin. Chem. 37:1618 (1991), Stenman et al., Cancer Res. 51:222 (1991) and WO 92/09136).

Serum PSA has become the most clinically useful tumor marker in prostatic disease. PSA is used to monitor the response to therapy and detect early relapses for prostate cancer. Prostate cancer is the most common cancer in men with an estimated 317,000 cases in 1996 in the United States. It is the second leading cause of death among men who die from neoplasia with an estimated 40,000 deaths per year. Prompt detection and treatment is needed to limit mortality caused by prostate cancer. When prostate cancer is treated with radiation, surgery or androgen-deprivation, the serum levels of PSA decrease. The level that they decrease to following therapy is correlated to prognosis and survival. When used following radical prostatectomy, the PSA level following successful treatment should decrease to zero, since the prostate is the only significant source of PSA. This allows PSA to be a very sensitive marker for tumor relapse following surgery. PSA has also been used to help stage patients. It has been shown that PSA level correlates with tumor volume, but is not accurate enough to independently stage the disease; however, PSA level is useful in combination with other clinical and pathological parameters (Partin et al., J. Urol. 152:1358-68 (1994)).

A major development in the use of PSA is its application to the early detection of prostate cancer prior to the appearance of clinical symptoms. Prior to the advent of the use of PSA, the digital rectal exam was used (DRE). This method was not very sensitive or specific. In addition, many tumors detected by DRE were too large to cure. The use of PSA in conjunction with DRE as an early detection method has been validated in several clinical studies. Typically, a cutoff of 4.0 ng/mL has been used as the upper limit of normal. Above this level, about 33% of biopsies are positive for cancer, while above 10 ng/mL, approximately 67% are positive for cancer. PSA levels above a value of 4.0 can be due to prostate inflammation or benign prostate hyperplasia (BPH). In men who have undergone negative prostate biopsies (no cancer), the prostate biopsy specimens usually reveal inflammation.

Patients with PSA below 4.0 ng/mL with a normal DRE are considered normal, while those above 10 ng/mL are considered more likely to have prostate cancer. Therefore, the gray zone is between 4 and 10 ng/mL.

Currently, men having a PSA level of 4.0 ng/mL or more are biopsied to determine if they have prostate cancer. However, the biopsy proves to be unnecessary for almost 2 out of 3 men with a PSA level from 4-10 ng/mL and 1 out of 3 men with a PSA level above 10 ng/mL.

In men with elevated PSA levels who have had a negative (no cancer) biopsy of the prostate, inflammation of the prostate is considered a significant contributor to the elevated PSA level. (Urology, 35 (6) 2000; 892-897, Okada K, Kojima M, Naya Y., Kamoi K, Yokoyama K, Takamatsu T, and Miki T. Correlation of Histologic Inflammation in Needle Biopsy Specimen with Serum PSA Levels in Men with Negative Biopsy for Prostate Cancer). In some of these men the inflammation may be related to infection. In men with symptomatic prostate inflammation or chronic prostatitis, positive bacterial cultures are found in less than 5% of cases. However, non-culturable microorganisms, or organisms that colonize the prostate may cause inflammation of the prostate, even though these men lack symptoms related to clinical prostatitis (Infections in Urology, 2000, March/April 31-38, Nickel J C, Chronic Prostatitis: An Infectious Disease?). In other words, the elevated PSA levels in some men may be secondary to inflammation resulting from bacteria or microorganisms present in the prostate.

The object of the present invention is to reduce the need for such biopsies in men suspected of having prostate cancer.

Another object of the present invention is to treat patients with elevated PSA levels.

### SUMMARY OF THE INVENTION

In accordance with the invention, the need for biopsies in men suspected of having prostate cancer can be reduced by determining if the level of the antigen indicator, such as PSA, can be reduced by the administration of an effective amount of an anti-inflammatory or a combination of an anti-inflammatory and an antibiotic.

Thus, one embodiment of the present invention is a method for reducing the prostate specific antigen level by administering an effective amount of an anti-inflammatory. Preferably, the anti-inflammatory is a non-steroidal anti-inflammatory. The method is preferably used when the PSA level is greater than 3 ng/mL, and preferably when the PSA level is from 4-10 ng/mL. The anti-inflammatory is preferably administered for 2-6 weeks and preferably for 4 weeks before the antigen level is retested.

Another embodiment of the present invention is a method for reducing the need for a biopsy in men having above normal prostate specific antigen levels, wherein the PSA level of a man is measured, when an above normal PSA level is obtained, an effective amount of an anti-inflammatory is administered and thereafter the measurement of the PSA level is repeated to determine if the level is normal. If the level after the retest is normal, a biopsy is no longer indicated. If the level is reduced, but still not normal, while a physician may still opt for a biopsy, research may show that reduced levels above the normal range may not be indicative of cancer and may prove to reduce the need for biopsies in many cases. Preferably, the anti-inflammatory is a non-steroidal anti-inflammatory. Moreover, preferably, the above normal PSA level is greater than 3 ng/mL. Furthermore, preferably, the anti-inflammatory is administered for 2-6 weeks, and preferably 4 weeks.

A further embodiment of the present invention is a method for reducing the need for biopsies in men suspected of having prostate cancer, wherein the level of an antigen indicator of prostate cancer is measured, and for an above normal level of the antigen indicator, an effective amount of an anti-inflammatory is administered. The measuring of the antigen indicator level is repeated to determine if the level is normal, whereby a biopsy is not indicated. Here again, if the level is reduced, but still not normal, while a physician may still opt for a biopsy, research may show that reduced levels above the normal range may not be indicative of cancer and may prove to reduce the need for biopsies in many cases. The anti-inflammatory is preferably a non-steroidal anti-inflammatory and the anti-inflammatory is preferably administered for 2-6 weeks, more preferably 4 weeks.

Another antigen that can be used for testing is prostatic acid phosphatase.

To treat men with elevated PSA levels associated with inflammation, the optimal treatment is with combination therapy of antibiotic and anti-inflammatory. The combination therapy provides treatment for cases where inflammation of the prostate is also associated with bacterial or microorganism colonization or infection.

The therapeutic effective amounts of the anti-inflammatory and antibiotic in accordance with this invention effectively treats both the inflammatory component and the bacterial/microorganism colonization or infectious component associated with the elevated PSA levels in men without prostate cancer.

The method of the invention comprises the administration of a therapeutically effective amount of a combination of an anti-inflammatory and an antibiotic, wherein the anti inflammatory agent is administered to alleviate symptoms of prostate inflammation unrelated to bacteria or microorganisms and the antibiotic is administered to reduce the inflammation associated with bacteria/microorganism colonization or infection. The administration can be either oral or parenteral.

The antibiotic is preferably selected from the group including Quinolones such as Nalidixic Acid, Floroquinolones such as Norfloxacin, Ciprofloxacin, Ofloxacin, Lomefloxacin, and Levofloxacin, Tetracyclines such as Doxycycline and Minocycline and combinations such as Trimethoprin and Sulfamethoxazole.

Another embodiment of the invention is a pharmaceutical composition comprising an amount of a combination of an anti-inflammatory and an antibiotic, said amount being effective to reduce the antigen indicator level of an patient having an elevated antigen indicator level. The antigen indicator is preferably the prostate specific antigen (PSA).

There are several different ways, described in more detail below, in which the therapeutic drug combination can be packaged or contained. The anti-inflammatory can be compounded into a single oral unit with the antibiotic, wherein the single oral unit is a tablet or a capsule.

The anti-inflammatory can also be formed into a first single oral unit, while the antibiotic can be formed into a second single oral unit. Any number of first single oral units can be placed in a first container, and any number of the second single oral units can be placed in a second container. The quantity of anti-inflammatory and antibiotic can be varied per each unit as well. Further, the first and second containers can be packaged together.

These and other objects and advantages of the present invention will be understood from the following detailed description and examples.

### DETAILED DESCRIPTION OF THE INVENTION

The present invention is particularly useful in conjunction with the conventional testing of men for an antigen level indicator of prostate cancer, and specifically for the testing of the PSA level in men.

Typically, a man going to a physician for a general physical and not complaining of any prostate related symptoms, is tested for his PSA level by his physician by sending a blood sample to a laboratory. If the results of the test show a PSA level of 3 ng/mL or less, no further action is normally indicated by the physician. However, for a test result of 4 ng/mL or greater, a physician will normally recommend that a biopsy be performed to determine if there is prostate cancer.

In accordance with the present invention, instead of proceeding directly to the biopsy, the physician will prescribe an anti-inflammatory for the patient or an anti-inflammatory in combination with an antibiotic for the patient. A list of suitable anti-inflammatories, the dosage of each and the period for which the anti-inflammatory should be administered is set forth below in Table I.

TABLE I

| NSAID | DOSE | |
|---|---|---|
| Diclofenac | 100-200 | mg/day in divided doses |
| Etodolac | 600-1200 | mg/day in divided doses |
| Fenoprofen | 600-2400 | mg/day in divided doses |
| Ibuprofen | 1.2-3.2 | grams/day in divided doses |
| Indomethacin | 50-200 | mg/day in divided doses |
| Ketoprofen | 200-300 | mg/day in divided doses |
| Mefenamic Acid | 500-1000 | mg/day in divided doses |
| Meloxicam | 7.5-15 | mg/day |
| Nambumetone | 1-2 | grams/day |
| Naproxen | 500-1500 | mg/day in divided doses |
| Oxaprozin | 1200-1800 | mg/day |
| Piroxicam | 20 | mg/day |
| Sulindac | 150-400 | mg/day in divided doses |
| Tolmetin Sodium | 600-1800 | mg/day in divided doses |
| Celecoxib | 100-800 | mg/day in divided doses |
| Rofecoxib | 25-100 | mg/day |

The above-listed dosage for each of the drugs should be administered for 2 to 6 weeks and preferably 4 weeks before retesting.

After taking the anti-inflammatory for 2-6 weeks, if the cause of the elevated PSA level is not prostate cancer, but rather an inflammation of the prostate, the PSA level will be reduced preferably to normal levels when retesting is performed after the 2-6 weeks.

This reduction in the PSA level will indicate to the physician that the cause for the elevated PSA level was not prostate cancer and will eliminate the need for a biopsy for that patient.

Each of the anti-inflammatory agents described above in the present invention can be used in combination with the antibiotics in parenteral or topical administration. In this invention, the anti-inflammatory and antibiotic are administered in combination separately or as one single combined pharmaceutical composition via parenteral or oral means. Preferably the anti-inflammatory and antibiotic are administered orally as a single combined pharmaceutical composition.

The anti-inflammatory and antibiotic compositions are generally administered in accordance with the current Physician's Desk Reference (PDR). (Medical Economics Co. Inc. of Oradell, N.J. 07649 53 edition, 1999).

The antibiotics and the dosages thereof are as follows:

| | |
|---|---|
| Quinolones: | |
| Nalidixic Acid | 2 grams/day in divided doses for 2 to 6 weeks and |
| | preferably 4 weeks |
| Floroquinolones | |
| Norfloxacin | 400 mg twice/day for 2 to 6 weeks and |
| | preferably 4 weeks |
| Ciprofloxacin | 250 to 500 mg twice/day for 2 to 6 weeks and |
| | preferably 4 weeks |
| Ofloxacin | 200 to 400 mg twice/day for 2 to 6 weeks and |
| | preferably 4 weeks |
| Lomefloxacin | 400 mg daily for 2 to 6 weeks and |
| | preferably 4 weeks |
| Levofloxacin | 250 to 500 mg daily for 2 to 6 weeks and |
| | preferably 4 weeks |
| Tetracyclines | |
| Doxycycline | 100 to 150 mg twice/day for 2 to 6 weeks and |
| | preferably 4 weeks |
| Minocycline | 100 mg twice/day for 2 to 6 weeks and |
| | preferably 4 weeks |
| Combinations | |
| Trimethoprin | 160 mg and Sulfamethoxazole 800 mg twice/day |
| | for 2 to 6 weeks and preferably 4 weeks |

The anti-inflammatory and antibiotic may be compounded into a single dosage form suitable for oral or parenteral administration. A tablet or capsule or caplets are particularly convenient forms for oral administration. Such compositions useful in the present invention are typically formulated with conventional pharmaceutical excipients, e.g., spray dried lactose and magnesiun stearate into tablets or capsules for oral administration. One or more of the active substances, with or without additional types of active agents, can be worked into tablets or dragee cores by being mixed with solid, pulverulent carrier substances, such as sodium citrate, calcium carbonate or dicalcium phosphate, and binders such as polyvinyl pyrrolidone, gelatin or cellulose derivatives, possibly by adding also lubricants such a magnesium stearate, sodium lauryl sulfate, “Carbowax” or polyethylene glycols. Of course, taste-improving substances can be added in the case of oral administration forms.

As further forms of administration, one can use plug capsules, e.g. hard gelatin, as well as closed softgelatin capsules comprising a softener or plasticizer, e.g. glycerine. The plug capsules contain the active substance preferably in the form of a granulate, e.g. in mixtures with fillers, such as lactose, saccharose, mannitol, starches such as potato starch or amylopectin, cellulose derivatives or highly dispersed silicic acids. In soft gelatin capsules, the active substance is preferably dissolved or suspended in suitable liquids, such as vegetable oils or liquid polyethylene glycols.

The active ingredient components used in accordance with the present invention may also be formulated into twice a day, once-a-day or even longer sustained release composition by conventional techniques well known in the art.

In place of oral administration, the active compounds may be administered parenterally. In such case, one can use a solution of the active substance, e.g., in sesame oil or olive oil.

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the subject being treated for any of the indications for the compounds of the invention indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

It is understood that the embodiments described hereinabove are merely illustrative and are not intended to limit the scope of the invention. It is realized that various changes, alterations, rearrangements and modifications can be made by those skilled in the art without substantially departing from the spirit and scope of the present invention.

1.-26. (canceled)

27. A method for determining the need for a biopsy in a man having an above-normal prostate specific antigen (PSA) level, said method comprising the steps of:
a) measuring the PSA level of the man a first time and determining that the PSA level is above-normal;
b) administering an effective amount of a combination of (i) an anti-inflammatory and (ii) an antibiotic for a period of time sufficient to reduce any inflammation of the prostate; and
c) measuring the PSA level a second time; and either:
i) determining that the PSA level is normal, in which case a biopsy is not performed; or
ii) determining that the PSA level is above-normal, in which case a biopsy is optionally performed.

28. The method according to claim 27, wherein the anti-inflammatory is a non-steroidal anti-inflammatory.

29. The method according to claim 27, wherein the above-normal PSA level is greater than 3 ng/mL.

30. The method according to claim 27, wherein the combination is administered for 2-6 weeks.

31. The method according to claim 27, wherein the antibiotic is selected from the group consisting of Quinolones, Floroquinolones, Tetracyclines and combinations thereof.

32. The method according to claim 27, wherein the antibiotic is selected from the group consisting of Nalidixic Acid, Norfloxacin, Ciprofloxacin, Ofloxacin, Lemofloxacin, Doxycycline, Minocycline, Trimethoprin and Sulfamethoxazole and combinations thereof.

33. The method according to claim 27, which comprises the steps of:
a) measuring the PSA level of the man a first time and determining that the PSA level is above-normal;
b) administering an effective amount of the combination to said man for a period of time deemed sufficient to reduce any inflammation of the prostate; and
c) measuring the PSA level a second time and determining that the PSA level is normal, in which case a biopsy is not performed.

34. The method according to claim 27, which comprises the steps of:
a) measuring the PSA level of the man a first time and determining that the PSA level is above-normal;
b) administering an effective amount of the combination to said man for a period of time deemed sufficient to reduce any inflammation of the prostate; and
c) measuring the PSA level a second time and determining that the PSA level is above-normal, in which case a biopsy is optionally performed.

35. A method for determining the need for a biopsy in a man suspected of having prostate cancer, said method comprising the steps of:
a) measuring the level of an antigen indicator of prostate cancer of the man a first time and determining that the level of the antigen indicator is above-normal;
b) administering an effective amount of a combination of (i) an anti-inflammatory and (ii) an antibiotic for a period of time sufficient to reduce any inflammation of the prostate; and
c) measuring the level of the antigen indicator a second time; and either:
i) determining that the level of the antigen indicator is normal, in which case a biopsy is not performed; or
ii) determining that the level of the antigen indicator is above-normal, in which case a biopsy is optionally performed.

36. The method according to claim 35, wherein the anti-inflammatory is a non-steroidal anti-inflammatory.

37. The method according to claim 35, wherein the combination is administered for 2-6 weeks.

38. The method according to claim 35, wherein the antibiotic is selected from the group consisting of Quinolones, Floroquinolones, Tetracyclines and combinations thereof.

39. The method according to claim 35, wherein the antibiotic is selected from the group consisting of Nalidixic Acid, Norfloxacin, Ciprofloxacin, Ofloxacin, Lemofloxacin, Doxycycline, Minocycline, Trimethoprin and Sulfamethoxazole and combinations thereof.

40. The method according to claim 35, which comprises the steps of:
a) measuring the level of an antigen indicator of prostate cancer of the man a first time and determining that the level of the antigen indicator is above-normal;
b) administering an effective amount of the combination to said man for a period of time deemed sufficient to reduce any inflammation of the prostate; and
c) measuring the level of the antigen indicator a second time and determining that the antigen indicator level is normal, in which case a biopsy is not performed.

41. The method according to claim 35, which comprises the steps of:
a) measuring the level of an antigen indicator of prostate cancer of the man a first time and determining that the level of the antigen indicator is above-normal;
b) administering an effective amount of the combination to said man for a period of time deemed sufficient to reduce any inflammation of the prostate; and
c) measuring the level of the antigen indicator a second time and determining that the antigen indicator level is above-normal, in which case a biopsy is optionally performed.
